(19) United States
(12) Patent Application Publication
(10) Pub. No.: US 2004/0009511 A1
(43) Pub. Date: Jan. 15, 2004

(54) Identification of compounds which stimulate bone formation using a cell-based screening assay targeting BMP signaling

(76) Inventors: Gregory R. Mundy, San Antonio, TX (US); Di Chen, San Antonio, TX (US); Ming Zhao, Helotes, TX (US)

Correspondence Address: MORRISON & FOERSTER LLP, 3811 VALLEY CENTRE DRIVE, SUITE 500, SAN DIEGO, CA, 92130-2332, US

(21) Appl. No.: 10/423,262

(22) Filed: Apr. 24, 2003

Related U.S. Application Data
(60) Provisional application No. 60/375,551, filed on Apr. 24, 2002.

(51) Int. Cl. C12Q 1/68; C07H 21/04; C07K 14/705; C12P 21/02; C12N 5/06

(52) U.S. Cl. 435/6; 536/23.5; 435/320.1; 435/69.1; 435/325; 530/350

(57) ABSTRACT

Methods and compositions for measuring the effects of molecules which stimulate BMP and Cbfa1 signaling are disclosed, wherein the response elements in the Smad family and Cbfa1 are utilized in an assay system to modulate the production of an assayable product of a reporter construct.

### TECHNICAL FIELD

The present invention relates to nucleic acid molecules and assay techniques for identifying compounds that affect bone morphogenetic protein (BMP) signaling pathways.

### BACKGROUND OF THE INVENTION

Bone morphogenetic proteins are a group of growth factors, which belong to the TGF-β superfamily. BMPs were originally identified from bone matrix using an ectopic bone formation assay (Urist, 1965; Wozney et al, 1988). Careful examination of the ectopic bone formation model indicates that it closely mimics the normal processes of endochondral bone formation (Wozney, 1992). The functions of BMP-2, BMP-6 and BMP-7 in ectopic bone formation and fracture repair have been well characterized (Wozney et al, 1988; Gitelman et al, 1994; Sampath et al, 1992). They induce ectopic bone formation and accelerate the healing process during fracture repair. Consistent with a role for BMPs in bone development and bone formation, studies of naturally occurring mutations have shown that different members of the BMP family may control the formation of different morphological features in the mammalian skeleton. For example, mutation in the BMP-5 gene is associated with a wide range of skeletal defects, including reductions in long bone width and the size of several vertebral processes and an overall lower body mass (Kingsley et al, 1992). Mutations in the growth differentiation factor 5 (Cartilage-derived Morphogenetic Protein−1/BMP-11) gene result in brachypodism in mice (Storm et al, 1994) and chondrodysplasia in humans (Thomas et al, 1996).

A number of in vitro studies also have demonstrated the importance of BMPs in osteoblast differentiation. BMP-2 stimulates osteoblast differentiation in primary osteoblastic cells (Harris et al, 1995; Chen et al, 1997) and in cell lines derived from osteogenic tissues (Yamaguchi et al, 1991; Takuwa et al, 1991; Thies et al, 1992). BMP-2 up-regulates BMP-2 and BMP-4 mRNA expression in primary osteoblastic cells, suggesting that BMP-2 acts as an autocrine as well as a paracrine factor during osteoblast differentiation (Harris et al, 1995; Chen et al, 1997). One of the mechanisms by which BMPs may stimulate osteoblast differentiation is through activating the osteoblast specific transcription factor, Cbfa1. Cbfa1 plays a critical role in osteoblast differentiation, bone development and postnatal bone formation. In homozygous Cbfa1 knockout mice, both the membranous bones of the skull and endochondral bones in the rest of the skeleton are absent (Komori et al, 1997). Heterozygous mutations of the Cbfa1 gene are found in humans with cleidocranial dysplasia (CCD) (Mundlos et al, 1997; Otto et al, 1997). BMPs may be important regulators of Cbfa1, as indicated by studies showing that BMP-7 and BMP-2 stimulate Cbfa1 expression in pluripotent mouse fibroblast C3H10T1/2 cells and in osteoblast precursor 2T3 cells (Ducy et al, 1997; Chen et al, 1998).

BMPs signal through serine/threonine kinase receptors, composed of type I and type II components. Both type I and type II BMP receptors are indispensable for signal transduction. During BMP signaling, the type II receptor kinase transphosphorylates the GS domain in type I receptor, which leads to activation of the type I receptor kinase. Three type I receptors have been shown to bind to BMP ligands, namely the type IA and IB BMP receptors (BMPR-IA and BMPR-IB) and type IA activin receptor (ActR-IA) (Koenig et al, 1994; ten Dijke et al, 1994). Recently, it has been shown that BMPR-IB plays a critical role in osteoblast commitment and differentiation in vitro and bone formation in vivo (Chen et al, 1998; Zhao et al, 2002).

The type I BMP receptor substrates include a recently identified protein family, the Smad proteins, that play a central role in the relay of BMP signals from the receptor to target genes in the nucleus. Smad1 (Hoodless et al, 1996), Smad5 (Nishimura et al, 1998) and Smad8 (Chen, Bhushan and Vale, 1997) are phosphorylated by BMP receptors in a ligand-dependent manner. These receptor-regulated Smads physically associate with the ligand-activated receptor complex and undergo phosphorylation at the C-terminus (Nishimura et al, 1998). After release from the receptor, Smad proteins associate with the related protein Smad4, which acts as a shared partner. This complex translocates into the nucleus and participates in gene transcription with other transcription factors. Smads can directly bind to DNA; however, the affinity is relatively low and interaction with sequence-specific DNA binding proteins is critical for the formation of a stable DNA-binding complex (Derynck et al, 1998). The first demonstration that Smads can directly bind to DNA was reported in drosophila (Kim et al, 1997). Vestigial, Labial and Ultrabithorax (Ubx) are decapentaplegic (dpp)-responsive genes. Mad, a drosophila homologue of Smad, was shown to directly bind to the enhancer of these genes, and GCCGnCGC (GCCG motif) was identified as the consensus binding site. Recent studies have showed that Smad1 can directly interact with the Cbfa1 protein (Hanai et al, 1999).

The response elements of BMP-2 and BMP-4 have been previously described (see supra), however, they have not been linked in the manner described here for identifying potential new drugs.

### SUMMARY OF THE INVENTION

In the present studies, a reporter construct is provided which contains multiple copies of the response elements of Smad1, 5 or 8, and Cbfa1. According to the present invention, this reporter construct will allow these Smad and Cbfa1 proteins to bind the DNA response elements, stabilize the protein-DNA complex and amplify BMP signaling. Clonal cell lines, in which the reporter construct is stably transfected, can be used to monitor BMP activity and as a cell-based screening assay to identify molecules which stimulate BMP signaling and bone formation.

The present application is directed to identifying the effects of the stimulation of the BMP signal transduction pathway. Unique combinations of response elements in target genes are utilized. For example, response elements for the BMP-2, BMP-4 and BMP-7 target genes have been linked to the firefly luciferase reporter. Further, the effects of compounds enhancing BMP-2 effects are measured.

A cell-based assay technique for identifying and evaluating the effects of BMP signaling is provided, comprising culturing a host cell line comprising a recombinant expression vector comprising a DNA sequence encoding the response elements of at least one Smad protein and at least one Cbfa1 protein, operatively linked to a reporter gene encoding an assayable product under conditions which permit expression of said assayable product, contacting the cultured cell line with at least one compound suspected of possessing bone enhancing activity, and identifying such compounds by their ability to modulate the expression of the reporter construct and thereby increase the production of the assayable product. The expression vector or nucleic acid molecule may optionally further comprise a promoter, e.g., osteocalcin basal promoter or a basal promoter of an osteoblast, chondroblast or chondrocyte-specific gene. And, the measurement of the production of said assayable product may be performed in the presence and absence of the compound.

This assay technique specifically identifies osteogenic compounds that stimulate BMP and Cbfa1 signaling pathways and subsequent bone formation. These osteogenic compounds display the capacity to increase the activity of the promoters of genes of members of the Smad and Cbfa1 families normally produced by, e.g., bone cells. Therefore, osteogenic compounds identified through the present methods are useful for treatment of bone deficit as the result of osteoporosis, segmental bone defects, bone fracture repair, prosthesis fixation, dental implantation, plastic surgery, or a disease associated with bone loss, cartilage injury or disorder, such as hyperparathyroidism, metastatic bone disease, or degenerative joint conditions, through the administration of a therapeutically effective amount of said compound.

Also provided in accordance with the present invention are isolated DNA sequences encoding the response elements of more than one of the Cbfa1 proteins and more than one Smad1, Smad5, or Smad8 protein (together hereinafter “Smad” proteins, unless otherwise indicated), said response elements may be operatively linked to a nucleotide sequence encoding an assayable product. Further, a system for identifying osteogenic compounds is provided comprising an expression vector comprising such response elements operatively linked to a reporter gene encoding an assayable product, and means for detecting the assayable product produced in response to exposure to an osteogenic compound.

The invention also includes compositions and kits containing them for use in these methods.

### BRIEF DESCRIPTION OF THE DRAWINGS

FIG. 1a presents a diagram of the reporter construct of a 9×SBE-6×OSE2-OC-luc reporter construct.

FIG. 1b presents the nucleotide sequence of a 9×SBE-6×OSE2-OC-luc reporter construct.

### DETAILED DESCRIPTION OF THE INVENTION

A cell-based assay technique for identifying and evaluating compounds, which stimulate BMP and Cbfa1 signaling and bone formation and enhancing compounds is provided, comprising culturing a host cell line comprising an expression vector comprising a DNA sequence encoding a combination of the response elements of more than one Smad protein (selected from Smad1, Smad5 and Smad8) and more than one Cbfa1 protein, operatively linked to a reporter gene encoding an assayable product under conditions which permit expression of said assayable product, contacting the cultured cell line with at least one compound suspected of possessing bone enhancing activity, and identifying such compounds by their ability to modulate the expression of the reporter construct and thereby increase the production of the assayable product. The expression vector may optionally further comprise a promoter, e.g., osteocalcin basal promoter and/or a basal promoter of an osteoblast, chondroblast or chondrocyte-specific gene. Without being bound by any theory regarding the mechanism of action, the combination and interaction of Smad1, Smad5 and/or Smad8 with Cbfa1 generates a stable complex and subsequent co-activation of target genes. This complex aids in stabilizing the protein-DNA complex and amplifying BMP signaling.

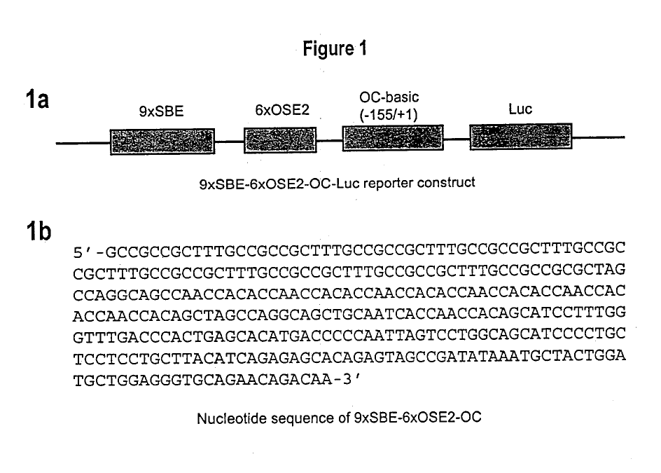

The homologous response elements of Smad are able to bind Smad1, Smad5 and Smad8 in the present invention. When BMP signaling is activated, any of the listed Smad proteins (1, 5 or 8) may be bound to the Smad response element (SBE) and stimulate the reporter gene. As provided above, FIG. 1b presents the nucleotide sequence of a 9×SBE-6×OSE2-OC-luc reporter construct, which includes 9 copies of a Smad response element (SBE).

The present invention is distinguished from other techniques for identifying bone-active compounds, as it specifically identifies chemical or biological compounds, agents, factors or other substances which stimulate bone morphogenetic protein (BMP) family signaling pathways (hereinafter “osteogenic compounds”). These osteogenic compounds are identified by their capacity to initiate signaling and/or increase the activity of BMP signaling pathways. When patients are treated with such chemical or biological compounds, the relevant Cbfa1 and Smad response elements will be stimulated to induce osteoblast differentiation and enhance bone growth or bone healing. Such compounds identified by this assay technique may be used for the treatment of osteoporosis, segmental bone defects, fracture repair, prosthesis fixation, dental implantation, plastic surgery, or a disease associated with bone loss, cartilage injury or disorder such as hyperparathyroidism, metastatic bone disease, or degenerative joint conditions.

Compounds that inhibit bone morphogenetic protein production or Smad binding in bone or cartilage also may be useful in clinical situations of excess bone formation, which occurs in such diseases as osteoblastic metastases or osteosclerosis of any cause. Such compounds also may be identified in accordance with the present invention.

Also provided in accordance with the present invention are isolated nucleic acid sequences encoding the response elements of more than one of the Smad proteins and more than one Cbfa1, and a system for identifying osteogenic compounds, comprising an expression vector comprising such response elements operatively linked to a reporter gene encoding an assayable product, and means for detecting the assayable product produced in response to exposure to an osteogenic compound.

The response elements of Smad and Cbfa1 are complex molecules that can be linked to reporter genes, such as, e.g., the firefly luciferase gene or another gene capable of detection through production of an assayable product, to create hybrid genes (also referred to as “reporter constructs”). When these reporter constructs are transfected into bone cells, osteogenic compounds, which activate the BMP and/or Cbfa1 signaling pathways can be identified by their capacity in vitro to increase production of an assayable product in cell lysates after cell culture with the compound. A particular non-limiting embodiment of a reporter construct contemplated by the present invention comprises multiple copies of Smad response elements and multiple copies of the Cbfa1 response element and firefly luciferase. Other suitable reporter genes contemplated by the present invention include chloramphenicol acetyl transferase (CAT) cDNAs, or cDNAs for other reporter genes such as β-galactosidase, green fluorescent protein, human growth hormone, alkaline phosphatase, β-glucuronidase, renilla luciferase (see U.S. Pat. Nos. 5,292,658 & 5,418,155), and the like. In addition to suitable promoter genes of the present invention recited below, any sort of reporter gene capable of detection through production of an assayable product, e.g., fluorescence, which is also capable of linkage to the present response elements, are suitable for the present invention.

The present invention isolates the Smad and Cbfa1 response element sequences and utilizes these response elements to make a reporter construct and transfect the reporter construct into cultured bone cells so that compounds could be identified, which are specifically directed to stimulating BMP signaling. Reporter constructs according to the present invention (see e.g., FIG. 1) are preferably comprised of multiple copies of the response elements of Smad and Cbfa1. The reporter construct preferably contains at least about 2 to about 15 copies of each response element. More preferably, the reporter construct comprises about 3 to about 12 copies of the response elements Smad and Cbfa1. The reporter construct of the present invention need not contain a similar number of copies of each response element, for example, the reporter construct may be comprised of about 9 copies of the sequence encoding the Smad1, Smad5, or Smad8 binding elements and about 6 copies of the sequence encoding Cbfa1 binding element (see FIG. 1a).

As provided, promoters may be included in the reporter constructs of the present invention. Examples of promoters suitable in the present invention include but are not limited to osteocalcin basal promoter or a basal promoter of an osteoblast, chondroblast or chondrocyte-specific gene. In a particular non-limiting embodiment, osteocalcin basal promoter represents a suitable promoter of the present invention.

The present invention isolates the DNA response elements for various genes and utilizes these DNA response elements to make a reporter construct and transfect this reporter into cultured bone cells so that compounds can be identified, which specifically stimulate BMP signal transduction pathways. Since it is known that the BMPs are produced by bone cells, a method for enhancing their signaling pathways specifically in bone should avoid systemic toxicity. This benefit is obtained by utilizing the unique tissue specific basal promoters for constructing the reporter genes, which are provided herein, and then using these reporters to identify compounds, which enhance BMP signaling in bone cells.

Accordingly, the present invention provides recombinant expression vectors comprising multiple nucleotide sequences encoding the response elements of Smad and Cbfa1. A variety of reporter vectors can be used to clone these response elements in those vectors. Commercially available reporter vectors that are useful for the present invention include but are not limited to, phRL-null, phRG-B, PGL3-Basic, pGEM-luc, pCAT3-Basic (Promega), Pluc-MSC (Strategene), pβgal-Basic, pECFP-1, pEGFP-1, pEYFP-1, and pSEAP2-Basic(Clontech) vector. The choice of the vector will depend upon the host cell type used, level of expression and the like.

The present invention further provides host cells engineered to contain and/or express a recombinant expression vector comprising the Smad and Cbfa1 response element nucleotide sequences. The cells that can be utilized in the present invention include primary cultures of fetal rat calvarial osteoblasts, and established bone cell lines available commercially (MC3T3-E1 cells, MG-63 cells, U20S cells, UMR106 cells, ROS 17/2.8 cells, SaOS2 cells and the like as provided in the catalog from the ATCC). Further preferable examples of cell lines for use in the present invention include multipotent fibroblast progenitor C3H10T1/2 cells, myoblast/osteoblast precursor C2C12 cells, osteoblast precursor 2T3 cells, and chondrocyte TMC-23 cells. In addition, a number of tumor cell lines also express BMPs, including the prostate cancer cell lines PC3, LNCAP, and DUI145, as well as the human cancer cell line HeLa. Thus, any of a number of cell lines will find use in the present invention and the use of an appropriate cell line will be a matter of choice for a particular embodiment.

In one aspect, cell lines of the present invention include transfected genes encoding more than one copy of the response elements of Smad and Cbfa1. Preferably, these cell lines are useful for monitoring BMP activity, as well as for a cell-based screening assay, to identify molecules, which stimulate BMP signaling and bone formation.

The methods provided herein are useful for identifying bone forming or bone enhancing compounds and compounds that stimulate BMP and Cbfa1 signaling whereby the cell or cell line described above may be contacted with a compound, which is suspected of possessing bone-enhancing activity. After contact, the assayable product resulting from such contact can be measured. According to one aspect of the present invention, the product produced in the presence of the compound is compared with product produced in the absence of the compound. Preferably, a compound that results in an increase in the production of the assayable product is identified as a bone-enhancing compound.

BMP signaling compounds can be examined in a variety of other assays that test specificity and toxicity. For instance, non-response elements and basal promoter can be linked to a reporter gene and transfected into an appropriate host cell. Cytotoxicity can be determined by visual or microscopic examination of the BMP signaling reporter construct-containing cells, for instance. Alternatively, nucleic acid and/or protein synthesis by the cells can be monitored. For in vivo assays, tissues may be removed and examined visually or microscopically, and optionally examined in conjunction with dyes or stains that facilitate histologic examination. In assessing in vivo assay results, it also may be useful to examine biodistribution of the test compound, using conventional medicinal chemistry/animal model techniques.

Further, methods are provided herein for treating an individual diagnosed or afflicted with or otherwise suspected of having a bone deficit or injury, comprising identifying a compound according to the methods described above and treating the individual with a therapeutically-effective amount of said compound. The treatment may be through any physiologically acceptable means known in the art. Determining dosage and treatment course may be determined through methods known in the art.

In addition, the present invention contemplates the use of additional compounds which stimulate BMP and Cbfa1 signaling pathways in osteoblastic and in other cell types. Non-limiting examples include BMP-2, BMP-4, BMP-6 and BMP-7, microtubule inhibitors (e.g., TN-16 & 2-methoxyestradiol), proteasome inhibitors (e.g., epoxomicin & PS-I), retinoic acids (e.g., all-trans & 9-cis), etc. Such compounds may readily be utilized in the present invention to provide comparable benefits.

As provided, methods presented herein are useful for treating disorders characterized by bone injury, loss, deficit, degeneration, and/or hypergeneration. As such, disorders for which the present invention will be beneficial include, but are not limited to: bone deficit as the result of osteoporosis, segmental bone defects, bone fracture repair, prosthesis fixation, dental implantation, plastic surgery, or a disease associated with bone loss, cartilage injury or disorder such as hyperparathyroidism, metastatic bone disease, or degenerative joint conditions.

The presently described reporter constructs can be packaged into diagnostic kits. Diagnostic kits may suitably include multiple copies of the Smad and Cbfa1 nucleotide sequences, which may be linked to reporter genes; alternatively, the multiple copies of the Smad and Cbfa1 nucleotide sequences may be not previously linked to the reporter genes prior to incorporation into the kit, and the ingredients for labeling may be included in the kit in separate containers so that the multiple copies of the Smad and Cbfa1 nucleotide sequences can optionally be linked to the reporter genes. Kits of the present invention may also contain other suitably packaged reagents and materials needed for the particular identification and characterization protocols, for example, standards, lysis buffers, as well as instructions for conducting the test.

Suitable kits may include multiple copies of the nucleic acid sequences encoding the response elements of Smad and Cbfa1 or representative vectors, cells or cell lines described above (all according to the present invention), useful for performing the provided methods for detecting suitable compounds and/or treating a disease or disorder.

While not seeking to be bound by any particular theory regarding the mechanism of action or functioning of the invention, it is believed that the reporter construct of the present invention, which contains multiple copies of the response elements of Smad 1, 5, or 8, and Cbfa1, will allow the Smad proteins and Cbfa1 to bind the DNA response elements, stabilize the protein-DNA complex and amplify BMP signaling as confirmed in the following Examples, because the combination and interaction of Smad and Cbfa1 and their interaction with the response elements generates a co-activation of target genes. However, the claimed invention is not limited to this mechanism. The presently described methods for identifying bone enhancing compounds and compounds which stimulate BMP signaling, as well as methods of treating individuals with compounds thus identified, can be effected through alternative mechanisms.

The following examples serve to illustrate certain preferred embodiments and aspects of the present invention and are not to be construed as limiting the scope thereof.

Experimental

In the experimental disclosure which follows, the following abbreviations apply: BMPs: Bone Morphogenetic Proteins; aFGF: Acidic Fibroblast Growth Factor; TGF-β: Transforming Growth Factor β; PTH: Parathyroid Hormone; eq (equivalents); M (Molar); mM (millimolar); μM (micromolar); N (Normal); mol (moles); mmol (millimoles); μmol (micromoles); nmol (nanomoles); kg (kilograms); gm (grams); mg (milligrams); μg (micrograms); ng (nanograms); L (liters); ml (milliliters); μl (microliters); vol (volumes); and ° C. (degrees Centigrade).

### EXAMPLE 1

Experimental Procedures

1. Immunoprecipitation

CHO cells were cultured with Dulbecco's modified Eagle's medium (DMEM), C3H10T1/2 multipotent fibroblast progenitor cells were cultured with RPMI 1604 medium, and C2C12 myoblast/osteoblast precursor cells were cultured with DMEM. The medium was purchased from Sigma (St. Louis, Mo.) supplemented with 10% fetal calf serum (FCS). CHO, C3H10T1/2 and C2C12 cells were plated in 10-cm culture dish at the density of 2×10⁶, 0.5×10⁶ and 1×10⁶ cells/dish and transfected with Myc-Smad1 and Flag-Cbfa1 expression plasmids (5 μg/well). Forty eight hours after transfection, cells were washed three times with phosphate-buffered saline (PBS) and solubilized in lysis buffer (150 mM NaCl, 1% Triton X-100, 0.5% doc and 50 mM Tris buffer, pH 7.5). The protease inhibitors aprotinin (10 mg/ml), leupeptin (10 mg/ml), and phenyl- methylsulfonyl fluoride (PMSF) (1 mM) were added to the lysis buffer. Cell lysates were centrifuged for 10 min at 4° C. at 10,000 g and incubated with anti-Myc antibody for 4 hours at 4° C., followed by immunoprecipitation with protein G-agarose (Boehringer Mannheim) at 4° C. overnight. Immunoprecipitates were washed with lysis buffer 5 times, added with 1× reducing buffer containing 0.5 M b-mercaptoethanol and boiled for 3 minutes. The immunoprecipitation samples were separated by sodium dodecyl sulphate -polyacrylamide gel electrophoresis (SDS-PAGE), transferred to nitrocellulose membrane, immunoblotted with anti-Flag or anti-Myc antibody and visualized with horse-radish peroxidase (HRP) coupled anti-mouse IgG antibody (Amersham) with an enhancement by enhanced chemiluminescence (ECL) detection kits (Amersham).

2. Formation of Reporter Construct

An oligonucleotide containing 9 copies of Smad1 response element (9×SBE) and 6 copies of Cbfa1 response element (6×OSE2) was synthesized in the DNA laboratory at University of Texas Health Science Center at San Antonio. The osteocalcin basal promoter (OC, −155/+1) was amplified by Polymerase Chain Reaction (PCR) using mouse genomic DNA as a template and cloned into pGL3 vector. An 9×SBE-6×OSE2 oligonucleotide was cloned upstream of osteocalcin basal promoter.

3. Stable Transfection

C2C12 and 2T3 cells were plated in 6-well culture plates at densities of 4×10⁵ and 2×10⁵ cells/well and cultured with DMEM or α minimal essential medium (α MEM) supplemented with 10% FCS for 24 hours. A 9×SBE-6×OSE2-OC-luc-reporter construct (2 μg/well) was co-transfected with pcDNA3 empty vector (0.2 mg/well) in C2C12 and 2T3 cells using lipofactamine plus reagents (Gibco/BRL). Forty eight hours later, G418 (200 μg/ml) was added to the culture plates. Four weeks after G418 selection, the G418 resistant cells were plated in 96-well plates at the density of 1 cell/well and cloned by limiting dilution method.

4. Luciferase Assay

C2C12 and 2T3 cells, in which 9×SBE-6×OSE2-OC-luc-reporter construct was stably transfected, were plated in 96-well culture plates at densities of 1×10⁴ and 0.5×10⁴ cells/well and cultured for 24 hours. The medium was changed to fresh medium supplemented with 2% FCS and different growth factors or hormone were added to culture medium for 24 hours or different compounds were added to culture medium for 2, 6, 12, 24 and 48 hours. The cell lysates were extracted and luciferase activities were determined by luciferase assay kit (Promega, Madison, Wis.) using a Luminometer.

### EXAMPLE 2

Smad1 and Cbfa1 Interactions and BMP Signaling Pathways

1. Smad1 Interacts with Cbfa1 in CHO, C3H10T1/2 and C2C12 Cells.

The interaction of Smad1 with Cbfa1 in C3H10T1/2, C2C12 and CHO cells was examined. Myc-Smad1 and Flag-Cbfa1 expression plasmids were transfected in these cells. To activate BMP signaling, a constitutively active BMPR-IA expression plasmid was co-transfected with Smad1 and Cbfa1 expression plasmids. Subsequently, a Cbfa1 protein was co-precipitated with Smad1 protein in these cells. These results demonstrate that Smad1 interacts with Cbfa1 upon activation of BMP signaling.

2. Cbfa1 is Required for Smad1 to Activate the Reporter Construct.

Multiple copies of a Smad1 response element (9×SBE) were cloned in front of a SV40 basal promoter (9×SBE-SV40-luc) and an osteocalcin basal promoter (9×SBE-OC-luc). The 9×SBE-OC-luc reporter construct responded to BMP-2 in a dose-dependent manner in both C2C12 and 2T3 cells and to the transfection of constitutively active BMPR-IA. In contrast, 9×SBE-SV40-luc reporter construct had no response to either BMP-2 treatment or transfection of constitutively active BMPR-IA. These results suggest that Cbfa1 is required for Smad1 to activate the target gene since there is an OSE2 site (Cbfa1 binding site) in osteocalcin basal promoter.

Further, the OSE2 site was mutated in the osteocalcin basal promoter in 9×SBE-OC-pGL3 reporter construct. After mutation, this reporter construct completely lost its responses to BMP-2 treatment and transfection of constitutively active BMPR-IA. These results indicate that Cbfa1 is required for Smad1 to activate the transcription of its target genes.

3. Creating a 9×SBE-6×OSE2-OC-luc Reporter Construct.

Further, a reporter construct which contains multiple copies of both Smad1 (SBE) and Cbfa1 (OSE2) response elements (9×SBE-6×OSE2-OC-luc) was made. The responsiveness of the new construct 9×SBE-6×OSE2-OC-luc and 9×SBE-OC-luc to BMP-2 was compared and it was found that this new construct responded to BMP-2 about 10-fold more than the construct 9×SBE-OC-luc.

4. Characterization of 9×SBE-6×OSE2-OC-luc Reporter Construct.

An examination was undertaken regarding the effects of the transfection of constitutively active BMPR-IA and Cbfa1 on a 9×SBE-6×OSE2-OC-luc reporter construct and it was found that both constitutively active BMPR-IA and Cbfa1 stimulated luciferase activity of this reporter construct. These results indicate that this reporter construct responds to BMP signaling and Cbfa1 activation. To determine whether Smad1 is required for Cbfa1 to activate this reporter construct, a mutant Smad1 (G419S) expression plasmid was co-transfected with Cbfa1 expression plasmid and it was found that expression of mutant Smad1 inhibits Cbfa1-induced luciferase activity by about 50%. This result indicates that Smad1 and Cbfa1 act together to co-activate the reporter construct. The dose-response and time-course effects of BMP-2 on the reporter construct were then examined and it was found that BMP-2 stimulates the luciferase activity of this reporter construct in a dose- and time-dependent manner.

5. Establishment of 2T3 and C2C12 Stable Cell Lines.

To establish C2C12 and 2T3 stable cell lines, a 9×SBE-6×OSE2-OC-luc reporter construct was stably transfected into these cells. A 9×SBE-6×OSE2-OC-luc reporter construct in a pGL3 vector and a pcDNA3 empty vector were co-transfected into C2C12 and 2T3 cells at 10:1 ratio. Forty eight hours after transfection, G418 was added to the cell culture at a concentration of 200 μg /ml. Four weeks after the selection, the cells were plated in 96-well plates at a density of 1 cell/well. Luciferase activity and BMP-2 responsiveness of the cells were tested when the single cell clones reached confluency.

Sixty four C2C12 cell stable clones were obtained and 21 of them were found to have luciferase activities and respond to BMP-2. Among these 21 clones, clone C-10 responds to proteasome inhibitor PS-I and epoxomicin in a dose-dependent manner at a maximal response of 6-8 fold increase in luciferase activity. To determine the stability of clone C-10, luciferase activity and BMP-2 responsiveness in every passage of clone C-10 was tested and found that luciferase activity was lost after passage 5, suggesting that clone C-10 is not a single cell clone. The clone C-10 was then subcloned from which 62 subclones were obtained. Clones C-10-10 and C-10-62 were found to contain luciferase activity and respond to BMP-2.

Similarly, 17 2T3 stable clones were obtained and two of them were found to have luciferase activity, clones C-15 and C-17. To determine the stability of clone C-17 and whether clone C-17 is a single cell clone, the luciferase activity and BMP-2 responsiveness was tested and found that clone C-17 maintains stability up to passage 13. Clone C-17 was then subcloned, from which 32 subclones were obtained. All 32 subclones demonstrated luciferase activity and responded to BMP-2. These results indicate that 2T3 clone C-17 is a stable clonal cell line.

6. Effects of BMPs on the Reporter Construct.

To determine the effects of BMPs on a 9×SBE-6×OSE2-OC-luc reporter construct, C2C12 clone C-10-62 and 2T3 clone C-17 were treated with different concentrations of BMP-2, BMP-4 and BMP-7 for 24 hours and changes in luciferase activity of this reporter construct were examined. In the assay using 2T3 clone 17, different concentrations of BMP-2 (see Table 1), BMP-4 (see Table 2) and BMP-7 (see Table 3) were added to the medium and cultured for 24 hours. At the end of the incubation, the cell lysates were extracted using lysis buffer and luciferase activities were measured using a luciferase assay kit (Promega).

It was found that BMP-2, BMP-4 and BMP-7 stimulated luciferase activity in a dose-dependent manner in both C2C12 C-10-62 and 2T3 C-17 cells (see Tables 1-3). The minimal effective doses of BMPs are 1-2 ng/ml for BMP-2, 0.1-0.2 ng/ml for BMP-4 and 15-30 ng/ml for BMP-7. BMP binding protein noggin was found to inhibit BMP-2-induced luciferase activity in a dose-dependent manner and 500 ng/ml of noggin completely blocked the effect of 100 ng/ml of BMP-2 on the reporter construct.

7. Effects of TGF-β, aFGF and PTH on the Reporter Construct.

To determine the specificity of BMPs on the activation of the reporter construct, the effects of TGF-β, aFGF and PTH on the reporter construct were examined. BMPs belong to the TGF-β superfamily, although BMPs and TGF-β seem to have different functions on osteoblast differentiation. Varied concentrations of TGF-β (0.01-10 ng/ml), aFGF (0.2-100 ng/ml) and PTH (0.2-100 ng/ml) were added to the C2C12 C-10-62 and 2T3 C-17 cells. The cells were incubated for 24 hours. The luciferase activities were measured after incubation. TGF-β was found to have a weak effect on the luciferase activity, at a maximum of 3-fold increase of the reporter construct, in 2T3 C-17 cells (see Table 5). The effect of TGF-β may be due to increased cell proliferation since no significant effect was found after TGF-β treatment when the luciferase activity was normalized to protein content. Further, aFGF and PTH were found to have no significant effects on the reporter construct in 2T3 C-17 cells, at a maximum of about less than 1-fold increase of the reporter construct (see Tables 4 and 6). Both TGF-β and aFGF were found to have no significant effects on the reporter construct in C2C12 C-10-62 cells. These results clearly indicate that this reporter construct specifically responds to BMP signaling.

8. Effects of Proteasome Inhibitors on the Reporter Construct.

To determine whether proteasome inhibitors stimulate the reporter construct, the effects of proteasome inhibitors PS-I and epoxomicin on the reporter construct, in C2C12 C-10-62 and 2T3 C-17 cells, were examined. It was found that proteasome inhibitors stimulated luciferase activity in a dose-dependent manner with maximal effect of about 4-5 fold increase in luciferase activity in both cell types (see Table 7).

It has been previously shown that proteasome inhibitors increase BMP-2 mRNA expression in human osteoblast MG-63 cells (Garrett et al, 2000). To determine whether proteasome inhibitors stimulated BMP-2 production in MG-63 cells, MG-63 cells were treated with different concentrations of epoxomicin for 1 and 5 hours, these cells were then washed away of epoxomicin and incubated with regular medium for additional 24 hours, and then 24 hour conditioned medium (CM) was collected. 2T3 C-17 cells were then treated with said CM for 48 hours. Using this CM to treat 2T3 C-17 cells, it was found that CM stimulated the reporter construct in a concentration-dependent manner. These results suggest that proteasome inhibitors can increase BMP production in MG-63 cells.

9. Effects of Microtubule Inhibitors on the Reporter Construct.

It has been found recently that microtubule inhibitors inhibit Smad1 binding to the microtubule, increase Smad1 nuclear translocation, and stimulate bone formation (Chen et al., unpublished data). Herein, the effects of microtubule inhibitors TN-16 and 2-methoxyestradiol on the reporter construct were examined. For example, a 2T3 cell clone 17 was treated with different concentrations of microtubule inhibitor TN-16 and 2-methoxyestradiol for 24 hours. It was found that both TN-16 and 2-methoxyestradiol stimulated the reporter construct in a dose-dependent manner in these cells (see Tables 8 & 9). TN-16 produced a maximal effect of a 5-fold increase in luciferase activity of the reporter construct.

10. Effects of Retinoic Acid on the Reporter Construct.

It has been reported that retinoic acids stimulate BMP-2 and -4 promoter activities in osteoblasts and in other cell types (Heller et al, 1999; Helvering et al, 2000). Herein the effects of retinoic acids on the reporter construct were examined. For this examination, 1.25 to 10 μM all-trans and 9-cis retinoic acids were added to C2C12 C-10-62 and 2T3 C-17 cells. For example, a 2T3 cell clone 17 was treated with different concentrations of retinoic acids for 24 hours. It was found that both all-trans and 9-cis retinoic acids stimulated the reporter construct in a dose-dependent manner, producing a maximal effect of a 5-fold increase of the luciferase activity in both cell types (see Table 10).

11. Effects of Other Compounds on the Reporter Construct.

To determine the specificity of the reporter construct, a series of unrelated compounds were tested on the reporter construct, such as protein kinase C activator 3-(N-acetylamino)-5-(N-decyl-N-methylamino)-benzyl alcohol (ADMB) (0.01-10 μM), protein kinase G activator 8-Bromo-cGMP and phosphodiesterase inhibitor pentoxifylline (0.01-10 μM) on the reporter construct. These compounds produced no significant effects on the reporter construct (less than 1-fold increase). These results indicate that the reporter construct only responds to compounds which activate BMP signaling.

All references cited herein are hereby incorporated by reference in their entireties, whether previously specifically incorporated or not. As used herein, the terms “a”, “an”, and “any” are each intended to include both the singular and plural forms and the term “or” is intended to refer to alternatives and combinations.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

Citation of the above publications or documents is not intended as an admission that any of the foregoing is pertinent prior art, nor does it constitute any admission as to the contents or date of these publications or documents.

TABLE 1

| | Luciferase Activity |
|---|---|
| BMP-2 (ng/ml) | (fold increase) |
| 0 | 0.067 |
| 0.39 | 0.106 (1.6) |
| 0.78 | 0.148 (2.2) |
| 1.56 | 0.200 (3.0) |
| 3.125 | 0.305 (4.6) |
| 6.25 | 0.492 (7.3) |
| 12.5 | 0.761 (11.4) |
| 25 | 1.235 (18.4) |
| 50 | 1.680 (25.1) |
| 100 | 2.102 (31.4) |

TABLE 2

| | Luciferase Activity |
|---|---|
| BMP-4 (ng/ml) | (fold increase) |
| 0 | 0.048 |
| 0.049 | 0.0745 (1.6) |
| 0.098 | 0.111 (2.3) |
| 0.195 | 0.141 (2.9) |
| 0.39 | 0.384 (8.0) |
| 0.78 | 0.597 (12.4) |
| 1.56 | 0.821 (17.1) |
| 3.125 | 1.064 (22.2) |
| 6.25 | 1.256 (26.2) |
| 12.5 | 1.534 (32.0) |
| 25 | 1.576 (32.8) |
| 50 | 1.887 (39.3) |
| 100 | 2.166 (45.1) |

TABLE 3

| | Luciferase Activity |
|---|---|
| BMP-7 (ng/ml) | (fold increase) |
| 0 | 0.067 |
| 1.95 | 0.076 (1.1) |
| 3.91 | 0.090 (1.3) |
| 7.81 | 0.108 (1.6) |
| 15.625 | 0.148 (2.2) |
| 31.25 | 0.216 (3.2) |
| 62.5 | 0.374 (5.6) |
| 125 | 0.813 (12.1) |
| 250 | 1.425 (21.3) |
| 500 | 2.224 (33.2) |
| 1000 | 3.404 (50.8) |

TABLE 4

| acid FGF (ng/ml) | Luciferase Activity |
|---|---|
| 0 | 0.078 |
| 0.195 | 0.06 |
| 0.39 | 0.057 |
| 0.78 | 0.058 |
| 1.56 | 0.051 |
| 3.125 | 0.052 |
| 6.25 | 0.054 |
| 12.5 | 0.051 |
| 25 | 0.052 |
| 50 | 0.058 |
| 100 | 0.075 |

BMP-2 (100 ng/ml): 31 fold increase

TABLE 5

| TGF-β (ng/ml) | Luciferase Activity |
|---|---|
| 0 | 0.047 |
| 0.0195 | 0.046 |
| 0.039 | 0.052 |
| 0.078 | 0.053 |
| 0.156 | 0.062 |
| 0.3125 | 0.08 |
| 0.625 | 0.09 |
| 1.25 | 0.102 |
| 2.5 | 0.156 |
| 5 | 0.077 |
| 10 | 0.081 |

BMP-2 (50 ng/ml): 29.6 fold increase

TABLE 6

| PTH (ng/ml) | Luciferase Activity |
|---|---|
| 0 | 0.078 |
| 0.195 | 0.05 |
| 0.39 | 0.053 |
| 0.78 | 0.047 |
| 1.56 | 0.05 |
| 3.125 | 0.05 |
| 6.25 | 0.048 |
| 12.5 | 0.051 |
| 25 | 0.053 |
| 50 | 0.053 |
| 100 | 0.055 |

BMP-2 (100 ng/ml): 31 fold increase

TABLE 7

| | Luciferase Activity |
|---|---|
| Epoxomicin (μM) | (fold increase) |
| 0 | 0.041 |
| 0.0156 | 0.041 (1.0) |
| 0.031 | 0.047 (1.1) |
| 0.0625 | 0.064 (1.6) |
| 0.125 | 0.098 (2.4) |
| 0.25 | 0.142 (3.5) |
| 0.5 | 0.153 (3.7) |
| 1 | 0.120 (2.9) |

TABLE 8

| | Luciferase Activity |
|---|---|
| TN-16 (μM) | (fold increase) |
| 0 | 0.023 |
| 0.039 | 0.022 (0.9) |
| 0.078 | 0.037 (1.6) |
| 0.156 | 0.036 (1.6) |
| 0.3125 | 0.038 (1.6) |
| 0.625 | 0.057 (2.5) |
| 1.25 | 0.081 (3.5) |
| 2.5 | 0.079 (3.4) |
| 5 | 0.111 (4.8) |
| 10 | 0.098 (4.2) |

TABLE 9

| | Luciferase Activity |
|---|---|
| 2-ME (μM) | (fold increase) |
| 0 | 0.05 |
| 0.625 | 0.052 |
| 1.25 | 0.059 (1.2) |
| 2.5 | 0.065 (1.3) |
| 5 | 0.102 (2.0) |
| 10 | 0.130 (2.6) |

2-ME (2-methoxyestradiol)

TABLE 10

Luciferase Activity

| 9-cis RA (μM) | All-trans RA | 9-cis RA |
|---|---|---|
| 0 | 0.045 | 0.045 |
| 1.25 | 0.113 (2.5) | 0.160 (3.6) |
| 2.5 | 0.142 (3.2) | 0.160 (3.6) |
| 5 | 0.164 (3.6) | 0.164 (3.6) |
| 10 | 0.219 (4.9) | 0.226 (5.0) |

References

Chen D, Harris M A, Rossini G, Dunstan C R, Dallas S L, Feng J Q, Mundy G R, and Harris S E (1997) Bone morphogenetic protein 2 (BMP-2) enhances BMP-3, BMP-4 and bone cell differentiation marker gene expression during the induction of mineralized bone matrix formation in cultures of fetal rat calvarial osteoblasts. Calcif Tissue Int 60:283-290.

Chen D, Ji X, Harris M A, Feng J Q, Karsenty G, Celeste A J, Rosen V, Mundy G R, and Harris S E (1998) Differential roles for BMP receptor type IB and IA in differentiation and specification of mesenchymal precursor cells to osteoblast and adipocyte lineages. J Cell Biol 142:295-305.

Chen Y, Bhushan A, and Vale W (1997) Smad8 mediates the signaling of the receptor serine kinase. Proc Natl Acad Sci USA 94:12938-12943.

Derynck R, Zhang Y, Feng X -H. (1998) Smads: transcriptional activator of TGF-βresponses. Cell 95:737-740.

Ducy P, Zhang R, Geoffroy V, Ridall A L, and Karsenty G (1997) Osf2/Cbfa1: A transcriptional activator of osteoblast differentiation. Cell 89:747-754.

Garrett I R, Gutierrez G, Chen D, Rossini G, Escobedo A, Esparza J, Horn D, Crews C M, and Mundy G R (2000) Specific inhibitors of the chymotryptic component of the proteasome are potent bone anabolic agents in vivo. J Bone Miner Res 15(Suppl1): S197.

Gitelman S E, Kobrin M S, Ye J Q, Lopez A R, Lee A, Derynck R (1994) Recombinant Vgr-1/BMP-6-expressing tumors induce fibrosis and endochondral bone formation in vitro. J Cell Biol 126:1595-1609.

Hanai J, Chen L, Kanno T, Ohtani-Fujita N, Kim W, Guo W, Imamura T, Ishidou Y, Fukuch M, Shi M, Stavnezer J, Kawabata M, Miyazono K, and Ito Y (1999) Interaction and functional cooperation of PEBP2/CBF with Smads: synergistic induction of the immunoglobulin germline Ca promoter. J Biol Chem 274:31577-31582.

Heller L C, Li Y, Abrams K L, and Rogers M B (1999) Transcriptional regulation of the BMP2 gene. J Biol Chem 274:1394-1400.

Helvering L M, Sharp R L, Ou X, and Geiser A G (2000) Regulation of the promoters for the human bone morphogenetic protein 2 and 4 genes. Gene 256: 123-138.

Hoodless P A, Haerry T, Abdollah S, Stapleton M, O'Connor M B, Attisano L, and Wrana J L (1996) MADR1, a MAD-related protein that functions in BMP2 signaling pathways. Cell 85:489-500.

Harris S E, Feng J Q, Harris M A, Ghosh-Choudhury N, Dallas M R, Wozney J M, and Mundy G R (1995) Recombinant bone morphogenetic protein 2 accelerates bone cell differentiation and stimulates BMP-2 mRNA expression and BMP-2 promoter activity in primary fetal rat calvarial osteoblast cultures. Mol Cell Differ 3:137-155.

Kim J, Johnson K, Chen H -J, Carroll S, and Laughon A. (1997) Drosophila Mad binds to DNA and directly mediates activation of vestigital by Decapentaplegic. Nature 388:304-308.

Kingsley D M, Bland A E, Grubber J M, Marker P C, Russell L B, Copeland N G, and Jenkins N A (1992) The mouse short ear skeletal morphogenesis is associated with defects in a bone morphogenetic member of the TGF beta superfamily. Cell 71:399-410.

Komori T, Yagi H, Nomura S, Yamaguchi A, Sasaki K, Deguchi K, Shimizu Y, Bronson R T, Gao Y, Inada M, Sato M, Okamoto R, Kitamura R, Yoshiki S, and Kishimoto T (1997) Targeted disruption of Cbfa1 results in a complete lack of bone formation owing to maturational arrest of osteoblasts. Cell 89:755-764.

Koenig B B, Cook J S, Wolsing D H, Ting J, Tiesman J P, Correa P E, Olson C A, Pecquet A L, Ventura F, Grant R A, CKomori T, Yagi H, Nomura S, Yamaguchi A, Sasaki K, Deguchi K, Shimizu Y, Bronson R T, Gao Y, Inada M, Sato M, Okamoto R, Kitamura R, Yoshiki S, and Kishimoto T (1997) Targeted disruption of Cbfa1 results in a complete lack of bone formation owing to maturational arrest of osteoblasts. Cell 89:755-764.

Mundlos S, Otto F, Mundlos C, Mulliken J B, Aylsworth A S, Albright S, Lindhout D, Cole W G, Henn W, Knoll J H M, Owen M J, Mertelsmann R, Zabel B U, and Olsen B R (1997) Mutations involving the transcription factor Cbfa1 cause cleidocranial dysplasia. Cell 89:773-779.

Nishimura R, Kato Y, Chen D, Harris S E, Mundy G R, and Yoneda T (1998) Smad5 and DPC4 are key molecules in mediating BMP-2-induced osteoblastic differentiation of the pluripo-tent mesenchymal precursor cell line C2C12. J Biol Chem 273:1872-1879.

Otto F, Thomell A P, Crompton T, Denze A, Gilmour K C, Rosewell I R, Stamp G W H, Beddington R S P, Mundlos S, Olsen B R, Selby P, and Owen M (1997) Cbfa1, a candidate gene for cleidocranial dysplasia syndrome, is essential for osteoblast differentiation and bone development. Cell 89:765-771.

Sampath T K, Maliakal J C, Hauschka P V, Jones W K, Sasak H, Tucker R F, White K H, Coughlin J E, Tucker M M, and Pang R H (1992) Recombinant human osteogenic protein-1 (hOP-1) induces bone formation in vivo with a specific activity comparable with natural bovine osteogenic protein and stimulates osteoblast proliferation and differentiation in vitro. J Biol Chem 267:20352-20362.

Storm E E, Huynh T V, Copeland N G, Jenkins N A, Kingsley D M, and Lee S J (1994) Limb alterations in brachypodism mice due to mutations in a new member of the TGF-β-superfamily. Nature 368:639-643.

Takuwa Y, Ohse C, Wang E A, Wozney J M, and Yamashita K (1991) Bone morphogenetic protein-2 stimulates alkaline phosphatase activity and collagen synthesis in cultured osteoblastic cells. Biochem Biophys Res Commun 174:96-101.

ten Dijke P, Yamashita H, Sampath T K, Reddi A H, Estevez M, Riddle D L, Ichijo H, Heldin C H, and Miyazono K (1994) Identification of type I receptors for osteogenic protein-1 and bone morphogenetic protein-4. J Biol Chem 269:16985-16988.

Thies R S, Bauduy M, Ashton B A, Kurtzberg L, Wozney J M, and Rosen V (1992) Recombinant human bone morphogenetic protein-2 induces osteoblastic differentiation in W-20-17 stromal cells. Endocrinology 130:1318-1324.

Thomas J T, Lin K, Nandedkar M, Camargo M, Cervenka J, and Luyten F P (1996) A human chondrodysplasia due to a mutation in a TGF-β superfamily member. Nature Genetics 12:315-317.

Urist M R (1965) Bone formation by autoinduction. Science 150:893-899.

Wozney J M (1992) The bone morphogenetic protein family and osteogenesis. Mol Reprod Dev 32:160-167.

Wozney J M, Rosen V, Celeste A J, Mitsock L M, Whitters M J, Kriz R, Hewick R, and Wang E A (1988) Novel regulators of bone formation: Molecular clones and activities. Science 242:1528-1534.

Yamaguchi A, Katagirl T, Ikeda T, Wozney J M, Rosen V, Wang E A, Kahn A, Suda T, and Yoshiki S (1991) Recombinant human bone morphogenetic protein-2 stimulates osteoblastic maturation and inhibits myogenic differentiation in vitro. J Cell Biol 113:681-687.

Zhao M, Harris S E, Horn D, Geng Z, Nishimura R, Mundy G R, and Chen D (2002) Type IB BMP receptor signaling is necessary for normal murine postnatal bone formation. J Cell Biol (in press)

1. An isolated nucleic acid molecule comprising
multiple copies of a nucleotide sequence encoding a Smad response element for Smad1, Smad 5 or Smad 8; and
multiple copies of a nucleotide sequence encoding a Cbfa1 response element.

2. The nucleic acid molecule of claim 1 further comprising a nucleotide sequence encoding an assayable product operatively linked to said response elements.

3. The nucleic acid molecule of claim 2 wherein the assayable product is firefly luciferase, chloramphenicol acetyl transferase (CAT), β-galactosidase, green fluorescent protein (GFP), human growth hormone, alkaline phosphatase, β-glucuronidase, or renilla luciferase.

4. The nucleic acid molecule of claim 1 wherein there are about 3 to about 12 copies of the sequence encoding the Smad response element and about 3 to about 12 copies of the sequence encoding the Cbfa1 response element.

5. The nucleic acid molecule of claim 4 wherein there are about 9 copies of the sequence encoding the Smad response element and about 6 copies of the sequence encoding the Cbfa1 response element.

6. The nucleic acid molecule of claim 1 further comprising a nucleotide sequence encoding a promoter.

7. The nucleic acid molecule of claim 6 wherein the promoter is osteocalcin basal promoter or a basal promoter of an osteoblast, chondroblast or chondrocyte-specific gene.

8. An isolated nucleic acid molecule comprising the nucleotide sequence of FIG. 1b.

9. A recombinant expression vector comprising the nucleic acid molecule of claim 1.

10. The recombinant expression vector of claim 9 further comprising a nucleotide sequence encoding an assayable product operatively linked to said response elements.

11. The recombinant expression vector of claim 10 wherein the assayable product is firefly luciferase, chloramphenicol acetyl transferase (CAT), β-galactosidase, green fluorescent protein (GFP), human growth hormone, alkaline phosphatase, β-glucuronidase, or renilla luciferase.

12. The recombinant expression vector of claim 9 wherein there are about 9 copies of the sequence encoding the Smad response element and about 6 copies of the sequence encoding the Cbfa1 response element.

13. The recombinant expression vector of claim 9 further comprising a nucleotide sequence encoding a promoter.

14. The recombinant expression vector of claim 13 wherein the promoter is osteocalcin basal promoter or a basal promoter of an osteoblast, chondroblast or chondrocyte-specific genes.

15. A recombinant host cell or cell line comprising the recombinant expression vector of claim 10.

16. The cell or cell line of claim 15 comprising multipotent fibroblast progenitor C3H10T1/2 cells, myoblast/osteoblast precursor C2C12 cells, osteoblast precursor 2T3 cells, or chondrocyte TMC-23 cells.

17. A method for identifying a bone-enhancing compound comprising:
contacting the cell or cell line of claim 15 with a compound suspected of possessing bone-enhancing activity; and
measuring the production of said assayable product in the presence and absence of the compound;
whereby a compound which results in an increase in the production of the assayable product is identified as a bone-enhancing compound.

18. A method for identifying a compound that stimulates bone morphogenetic protein (BMP) or Cbfa1 signaling comprising:
contacting the cell or cell line of claims 15 with a compound suspected of possessing BMP signaling stimulation; and
measuring the production of said assayable product in the presence and absence of the compound;
whereby a compound which results in an increase in the production of the assayable product is identified as a compound that stimulates BMP or Cbfa1 signaling.

19. The method of claim 18 wherein the BMP is BMP-2, BMP-4 or BMP-7.

20. A method for treating an individual suspected of having a bone deficit comprising:
identifying a compound according to the method of claim 17; and
treating said individual with a therapeutically-effective amount of said compound.

21. The method of claim 20 wherein the bone deficit is the result of osteoporosis, segmental bone defects, bone fracture repair, prosthesis fixation, dental implantation, plastic surgery, or a disease associated with bone loss, cartilage injury, or a bone disorder.

22. The method of claim 21 wherein the bone disorder is hyperparathyroidism, metastatic bone disease or degenerative joint conditions.

23. A diagnostic kit, comprising the nucleic acid molecule of claim 1 and instructions therefore.

24. A diagnostic kit, comprising the recombinant expression vector of claim 9 and instructions therefore.

25. A diagnostic kit, comprising the cell or cell line of claim 15 and instructions therefor.

26. The diagnostic kit of claim 23 further comprising an assayable product operatively linked to said response elements.

27. The diagnostic kit of claim 24 further comprising an assayable product operatively linked to said response elements.

28. The diagnostic kit of claim 25 further comprising an assayable product operatively linked to said response elements.
